(19) United States
(12) Patent Application Publication
(10) Pub. No.: US 2022/0059234 A1
(43) Pub. Date: Feb. 24, 2022

(54) INDIVIDUALIZED RISK SCORE INTERPRETATION AND DECISION TRAJECTORY COMPARISON

(71) Applicant: KONINKLIJKE PHILIPS N.V., EINDHOVEN (NL)

(72) Inventors: CLAIRE YUNZHU ZHAO, BOSTON, MA (US); KRISTEN TGAVALEKOS, CAMBRIDGE, MA (US); SHREYAS RAJ RAVINDRANATH, PHOENIX, AZ (US)

(21) Appl. No.: 17/519,633

(22) Filed: Nov. 5, 2021

Related U.S. Application Data
(63) Continuation-in-part of application No. PCT/EP20/64111, filed on May 26, 2021.
(60) Provisional application No. 63/030,442, filed on May 27, 2020.

(51) Int. Cl. G16H 50/30; G16H 50/20

(52) U.S. Cl. CPC G16H 50/30; G16H 50/20

(57) ABSTRACT

A system and for interpreting a risk score and comparing a decision trajectory are described. The system includes: a memory adapted to store: input features comprising clinical measurements of a patient; engineered features; a trained risk score computational model comprising instructions; a defined subgroup of patients; and a processor. The instructions, when executed by the processor causes the processor to: train a feature importance computational model using the defined subgroup of patients, the engineered features, and the risk score; determine a feature importance value using the trained feature importance computational model, the feature importance value comprising a contribution of a risk score; and provide a decision trajectory for the patient, or individualized feature outcomes for the patient over time, or both

### CROSS REFERENCE TO RELATED APPLICATIONS

The present application is a continuation-in-part of International Patent Application No. PCT/EP2021/064111 filed on May 26, 2021 which claims the benefit of Provisional Application No. 63/030,442 filed May 27, 2020. The present application is in accordance with the conditions and requirement of 35 U.S.C. § 120, and claims priority under 35 U.S.C. § 365(c) to International Patent Application No. PCT/EP2021/064111. The entire disclosure of International Patent Application No. PCT/EP2021/064111 is hereby specifically incorporated by reference in its entirety.

### BACKGROUND

Clinical decision support tools are designed to provide targeted and relevant information healthcare providers at key times during care. These tools guide medical diagnosis and inform therapeutic decisions, and have been shown to improve the performance of healthcare providers. Many current clinical decision support tools comprise machine learning (ML)-based clinical support systems. These ML-based tools have been shown to out-perform rule-based systems in predicting patient outcomes.

Some ML-based tools comprise patient-specific risk scores, indicating a risk of an outcome in view of one or more input features. The interpretation of machine-learning risk scores useful in making and supporting clinical decisions and transitions of care, and thus quantifying the level of certainty in a risk score prediction can reduce false alarm rates and further encourage clinicians' interpretation.

To finalize a clinical decision, it is also useful for clinicians to contrast a particular patient against different subgroups of patients who received different clinical decisions, such as different diagnosis, subtype of diseases, treatment, and care facilities. Moreover, it is useful for clinicians to understand what factors impact the risk score of a particular patient and across patient subgroups.

Information including risk scores enable clinicians to form the underlying (patho)-physiological context of the patient and associate the context in relation to the risk score. In addition, risk scores can be used establish causality of underlying biological processes to the more general deterioration represented by the risk score, by bridging the link between feature value and risk score.

Among other drawbacks, known systems and methods do not allow comparisons across multiple patient subgroups that are individualized and are in the context of the risk score.

What is needed, therefore, is a method and system that overcomes at least the drawbacks of known methods and systems described above.

### SUMMARY

According to an aspect of the present disclosure, a method of interpreting a risk score and comparing a decision trajectory is described. The method comprises: providing input features comprising clinical measurements of a patient; generating engineered features from the input features; providing the engineered features to a trained risk score computational model to determine a risk score of the patient; defining a subgroup of patients; training a feature importance computational model using the subgroup of patients, the engineered features, and the risk score; determining a feature importance value using the trained feature importance computational model; and providing decision trajectories for the patient, or individualized feature importance values for a patient over time, or both. Notably, feature importance values may be referred to as feature contributions.

According to another aspect of the present disclosure, a system for interpreting a risk score and comparing a decision trajectory is disclosed. The system comprises: a memory adapted to store: input features comprising clinical measurements of a patient; engineered features; a trained risk score computational model comprising instructions; a defined subgroup of patients; and a processor. The instructions, when executed by the processor causes the processor to: train a feature importance computational model using the defined subgroup of patients, the engineered features, and the risk score; determine a feature importance value using the trained feature importance computational model; and provide a decision trajectory for the patient, or individualized feature importance values for a patient over time, or both.

According to another aspect of the present disclosure, a tangible, non-transitory computer readable medium that stores a computational model comprising instructions is described. The tangible, non-transitory computer readable medium stores input features comprising clinical measurements of a patient; engineered features; a trained risk score computational model comprising instructions; a defined subgroup of patients. When executed by the processor, the instructions cause the processor to: train a feature importance computational model using a subgroup of patients, the engineered features, and a risk score from the trained risk score computational model; determine a feature importance value using the trained feature importance computational model; and provide a decision trajectory for the patient, or individualized feature importance values for a patient over time, or both.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system, according to a representative embodiment.

FIG. 2 is a simplified flow diagram showing a method, according to a representative embodiment.

FIG. 3 flow diagram showing a method of determining decision trajectories and feature contributions, according to a representative embodiment.

FIGS. 4A-4D are graphs showing feature values, feature contributions, and decision trajectories at a particular point in time according to a representative embodiment.

FIGS. 5A-5C are graphs showing feature values and feature contributions at over a particular time period, according to a representative embodiment.

FIGS. 6A-6E are graphs showing feature values and feature contributions at over a particular time period, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

By the present teachings, and among other improvements to the technical field, the risk score algorithms of representative embodiments described herein foster interpretations of features not only at a particular point in time (e.g., a clinical decision point), but also over a determined period of time.

Furthermore, and as will become clearer as the present description describes, the resultant outputs provided according to various representative embodiments facilitate an understanding of what underlying clinical measurements drive a risk score up and down.

Moreover, as will also become clearer as the present description continues, the decision pathways of particular patient at a given time can be compared to other patient data, including, for example, different spans of times in past history of the same patient. This helps clinicians to determine if patient's physiological state has changed

In one aspect, feature contributions are selectively added to provide the risk score for a patient. Beneficially, this allows a clinician to determine the overall risk scores based on features provided by the M/L, models of the present teachings.

Moreover, and as described more fully below, the methods and systems of the present teachings allow comparisons across multiple patient subgroups that are individualized and are in the context of the risk score. As will become clearer as the present description continues, selection of subgroups allows for the comparison of a particular patient against clinical sub-types subgroups with different diagnosis, subtype of diseases, treatment, and care facilities. These comparisons according to various representative embodiments, help clinicians weigh different clinical decisions for the patients

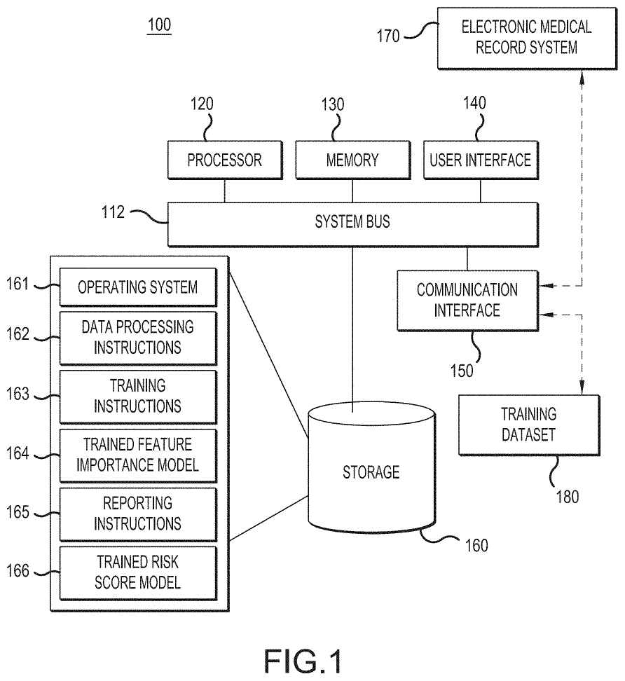

FIG. 1 is a simplified schematic representation of a patient risk score analysis system 100 in accordance with a representative embodiment.

According to a representative embodiment, and as described more fully below, the patient risk score analysis system 100 comprises a processor 120 adapted to execute instructions stored in memory 130 or in storage 160, or both, or otherwise process data to, for example, perform one or more steps of a method in accordance with representative embodiments described herein.

The processor 120 may take any suitable form, including but not limited to a microprocessor, microcontroller, multiple microcontrollers, circuitry, field programmable gate array (FPGA), application-specific integrated circuit (ASIC), a single processor, or plural processors. More generally, the term “processor” as used herein encompasses an electronic component able to execute a program or machine executable instructions, such as those described below. References to a computing device comprising “a processor” should be interpreted to include more than one processor or processing core, as in a multi-core processor. A processor may also refer to a collection of processors within a single computer system or distributed among multiple computer systems, such as in a cloud-based or other multi-site application. The term computing device should also be interpreted to include a collection or network of computing devices each including a processor or processors. Programs have software instructions performed by one or multiple processors that may be within the same computing device or which may be distributed across multiple computing devices.

As described more fully below, the processor 120 has access to an artificial intelligence (AI) engine, which may be implemented as software that provides AI and applies machine-learning described herein. The AI engine, which provides computational models described below, may reside in any of various components in addition to or other than storage 160 or memory 130, or both, or in an external server, and/or a cloud, for example. When the AI engine is implemented in a cloud, such as at a data center, for example, the AI engine may be connected to the processor 120 via the internet using one or more wired and/or wireless connection(s). The AI engine may be connected to multiple different computers including the processor 120, so that the artificial intelligence and machine-learning described below in connection with various representative embodiments are performed centrally based on and for a relatively large set of medical facilities and corresponding subjects at different locations. Alternatively, the AI engine may implement the artificial intelligence and the machine-learning locally to the processor 120, such as at a single medical facility or in conjunction with a single patient monitoring system.

The memory 130 can take any suitable form, including a non-volatile memory and/or RAM. The memory 130 may include various memories such as, for example L1, L2, or L3 cache or system memory. As such, the memory 130 may include static random access memory (SRAM), dynamic RAM (DRAM), flash memory, read only memory (ROM), or other similar memory devices. The memory can store, among other things, an operating system. The RAM is used by the processor for the temporary storage of data. According to a representative embodiment, an operating system may contain code which, when executed by the processor 120, controls operation of one or more components of patient risk score analysis system 100. It will be apparent that, in embodiments where the processor 120 implements one or more of the functions described herein in hardware, the software described as corresponding to such functionality in other embodiments may be omitted. The memory 130 may include a main memory and/or a static memory, where such memories may communicate with each other via the system bus 112. The memory 130 stores instructions used to implement some or all aspects of methods and processes described herein. The memory 130 may store various types of information, such as software algorithms, which serves as instructions, which when executed by a processor cause the processor to perform various steps and methods according to the present teachings.

User interface 140 may include one or more devices for enabling communication with a user. The user interface can be any device or system that allows information to be conveyed and/or received, and may include a display, a mouse, and/or a keyboard for receiving user commands. In some embodiments, user interface 140 may include a command line interface or graphical user interface that may be presented to a remote terminal via communication interface 150. The user interface may be located with one or more other components of the system, or may located remote from the system and in communication via a wired and/or wireless communications network.

Communication interface 150 may include one or more devices for enabling communication with other hardware devices. For example, communication interface 150 may include a network interface card (NIC) configured to communicate according to the Ethernet protocol. Additionally, communication interface 150 may implement a TCP/IP stack for communication according to the TCP/IP protocols. Various alternative or additional hardware or configurations for communication interface 150 will be apparent.

Storage 160 may include one or more machine-readable storage media such as various types of ROM and RAM noted above, magnetic disk storage media, optical storage media, flash-memory devices, or similar storage media. In various embodiments, storage 160 may store instructions for execution by processor 120 or data upon which processor 120 may operate. For example, storage 160 may store an operating system 161 for controlling various operations of patient risk score analysis system 100.

The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art.

The memory 130 and the storage 160 are tangible storage mediums for storing data and executable software instructions, and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 130 and storage may store software instructions and/or computer readable code that enable performance of various functions. The memory 130 and storage 160 may be secure and/or encrypted, or unsecure and/or unencrypted. The memory 130 and storage 160 store data and executable instructions used to implement some or all aspects of methods and processes described herein. Notably, the storage 160 can be foregone, and all data and executable instructions can be stored in memory 130.

It will be apparent from the present description, that various information described as stored in storage 160 may be additionally or alternatively stored in memory 130. In this respect, memory 130 may also be considered to constitute a storage device and storage 160 may be considered a memory. Various other arrangements will be apparent. Further, memory 130 and storage 160 may both be considered to be non-transitory machine-readable media. As used herein, the term non-transitory will be understood to exclude transitory signals but to include all forms of storage, including both volatile and non-volatile memories.

More generally, “memory” and “storage” are examples of computer-readable storage media, and should be interpreted as possibly being multiple memories or databases. The memory 130 or storage 160 may, for instance, be multiple memories or databases local to the computer, and/or distributed amongst multiple computer systems or computing devices. Furthermore, the memory 130 and the storage 160 comprise a computer readable storage medium that is defined to be any medium that constitutes patentable subject matter under 35 U.S.C. § 101 and excludes any medium that does not constitute patentable subject matter under 35 U.S.C. § 101.

While the patient risk score analysis system 100 is shown as including one of each described component, the various components may be duplicated in various embodiments. For example, the processor 120 may include multiple microprocessors that are configured to independently execute the methods described herein or are configured to perform steps or subroutines of the methods described herein such that the multiple processors cooperate to achieve the functionality described herein. Further, where one or more components of patient risk score analysis system 100 is implemented in a cloud computing system, the various hardware components may belong to separate physical systems. For example, processor 120 may include a first processor in a first server and a second processor in a second server. Many other variations and configurations are possible.

According to a representative embodiment, and as alluded to above, the storage 160 of patient risk score analysis system 100 may store one or more algorithms, modules, and/or instructions to carry out one or more functions or steps of the methods described or otherwise envisioned herein. For example, the patient risk score analysis system 100 may comprise, among other instructions or data, an electronic medical record system 170, and a training data set 180. As shown, the storage 160 illustratively comprises an operating system, data processing instructions 162, training instructions 163, a trained feature importance computational model 164, reporting instructions 165, and a trained preliminary risk score computational model 166.

According to a representative embodiment, the electronic medical record system 170 is an electronic medical records database from which the plurality of features may be obtained or received. The electronic medical records database may be a local or remote database and is in communication the patient risk score analysis system 100. According to a representative embodiment, the patient risk score analysis system comprises an electronic medical record database or system, which is optionally in direct and/or indirect communication with patient risk score analysis system 100.

According to a representative embodiment, the patient risk score analysis system 100 comprises a training data set 180. The training data or input features comprises clinical features of the particular patient as measured by the clinician, engineered features based on the clinical features and a preliminary risk score. As described more fully below, these training data are the input parameters used to train feature importance engine. In addition, the training data set 180 may comprise medical information about each of the patients, including but not limited to demographics, physiological measurements such as vital data, physical observations, and/or diagnosis, among many other types of medical information.

According to a representative embodiment, data processing instructions 162 direct the patient risk score analysis system 100 to retrieve and process input data which is used to train the feature importance model and the risk model as described more fully below. The data processing instructions 162 direct the patient risk score analysis system 100 for example, to receive or retrieve input data or medical data to be used by the system as needed, such as from electronic medical record system 170 among many other possible sources. As described above, the input data can comprise a wide variety of input types from a wide variety of sources.

According to a representative embodiment, the data processing instructions 162 also direct the system to process the input data to generate a plurality of features related to medical information for a plurality of patients, which are used to train the classifier. This can be accomplished by a variety of embodiments for feature identification, extraction, and/or processing. The outcome of the feature processing is a set of features related to risk analysis for a patient, which thus comprises a training data set that can be utilized to train the trained feature importance computational model 164.

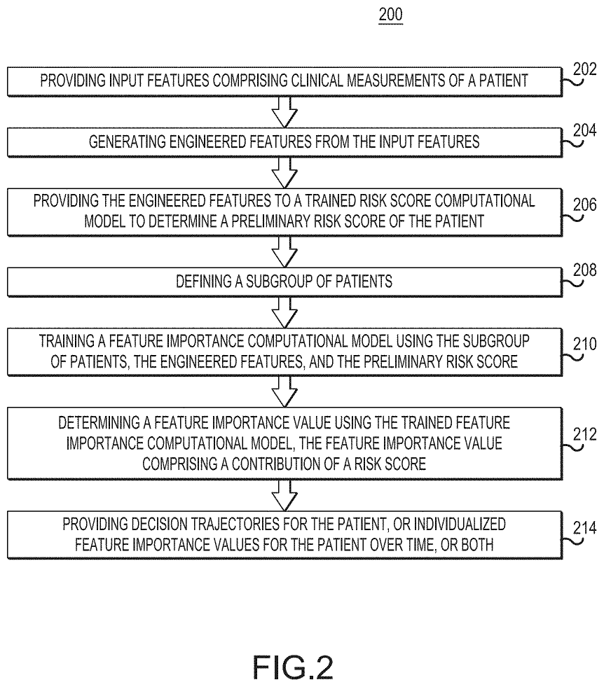

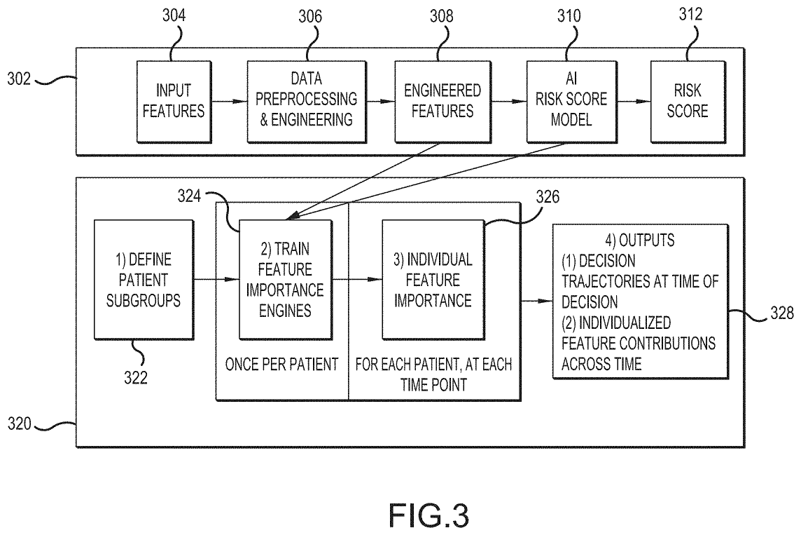

According to a representative embodiment, training instructions 163 direct the system to utilize the process data to train the trained feature importance computational model 164 and the trained preliminary risk score computational model 166. The trained feature importance computational model 164 and the trained preliminary risk score computational model 166 can be any machine learning algorithm, classifier, or model sufficient to utilize the type of input data provided, and to generate feature importance values and risk scores, respectively. Notably, and as described more fully below, the training instructions 163, the trained feature importance computational model 164 and the trained preliminary risk score computational model 166 provide machine-learning and resultant trained AI models comprising instructions, which when executed by the processor 120, cause the processor 120 to carry out the steps of the respective trained feature importance computational model 164, and the trained AI models to provide desired results. For example, and as described below in connection with FIGS. 2-3, by carrying out the steps of the trained feature importance computational model 164 and trained preliminary risk score computational model 166, feature importance values and risk scores, respectively, are provided, and are used to provide decision trajectories at a particular time and feature contributions over time.

According to a representative embodiment, reporting instructions 165 direct the system to generate and provide a report to a user via a user interface comprising a generated risk score range. According to a representative embodiment, the risk score range comprises the initial score with an indication of the calculated risk score confidence interval. According to a representative embodiment, the system also presents to the user via the user interface 140 one or more of the identified one or more missing features.

According to a representative embodiment, the reporting instructions 167 direct the system to display the report on a display of the system. The display may comprise information about the patient, the parameters, the input data for the patient, and/or the patient's risk. Other information is possible. Alternatively, the report may be communicated by wired and/or wireless communication to another device. For example, the system may communicate the report to a mobile phone, computer, laptop, wearable device, and/or any other device configured to allow display and/or other communication of the report.

More generally, after being trained, the computational models of the representative embodiments may be stored as executable instructions in memory 130, for example, to be executed by the processor 120. Furthermore, updates to the computational models may also be provided to the patient risk score analysis system 100 and stored in memory 130. Finally, and as will be apparent to one of ordinary skill in the art having the benefit of the present disclosure, according to a representative embodiment, the computational models may be stored in a memory and executed by a processor that are not part of the patient risk score analysis system 100, but rather are connected to the system through an external link (e.g., a known type of internet connection). Just by way of illustration, the computational models may be stored as executable instructions in a memory, and executed by a server that is remote from the patient risk score analysis system 100. When executed by the processor in the remote server, the instructions cause the processor to carry out the various methods in accordance with various representative embodiments described more fully herein.

FIG. 2 is a simplified flow diagram showing a method 200 of interpreting a risk score and comparing between decision trajectories from the patient subgroups, in accordance with a representative embodiment. The method 200 is contemplated to be carried out using patient risk score analysis system 100. Specifically, and in accordance with various embodiments of the present teachings, the method 200 described herein may be implemented using a hardware computer system such as patient risk score analysis system 100 that executes software programs. Further, in representative, non-limiting embodiments, implementations can include distributed processing, component/object distributed processing, and parallel processing. Virtual computer system processing may implement one or more of the methods or functionalities as described herein, and a processor described herein may be used to support a virtual processing environment. Certain aspects and details of the patient risk score analysis system 100 described in connection with FIG. 1 are common to the method 200 of FIG. 2, and may not be repeated to avoid obscuring the description of the method 200.

At 202, the method begins with providing input features comprising clinical measurements of a patient. These measurements are generally those taken by a clinician from a patient. By way of illustration, the input features may be stored in the electronic medical record system 170 or in storage 160 and include vital signs, for example, blood pressure, temperature, oxygen saturation (SpO2) of the patient. As described more fully below, these clinical measurements are feature values and provide one part of the training data set 180, and may be used in the trained feature importance computational model 164 and the trained preliminary risk score computational model 166 to ultimately provide decision trajectories at a particular point in time, and feature contributions over time.

At 204, the method continues with generating engineered features from the input features generated at 202. The generation of engineered features is carried by the processor 120 by executing instructions stored in the patient risk score analysis system 100, for example in memory 130 or storage 160. Notably, at 204 clearly erroneous measurements are discarded as well. Just by way of illustration, the engineered features may comprise trends of a feature (e.g., blood pressure, temperature, oxygen saturation (SpO2)), temporal characteristics of a feature, and clinical composite scores (e.g., shock ratio, ejection fraction and fluid accumulation). As described more fully below, engineered features provide another part of the training data set 180, and may be used in the trained feature importance computational model 164 and the trained preliminary risk score computational model 166 to ultimately provide decision trajectories at a particular point in time, and feature contributions over time.

At 206, the method comprises providing the engineered features to a trained preliminary risk score computational model 166 to determine a preliminary risk score of the patient. Notably, this trained preliminary risk score computational model 166 may be as described in the parent application. The trained preliminary risk score computational model 166, for example, stored in storage 160 as instructions, which when executed by the processor 120, causes the processor 120 to determine the preliminary risk score according to a representative embodiment.

At 208, the method comprises defining a subgroup of patients. As also described below in connection with FIG. 3, in accordance with a representative embodiment, subgroups of interest to a clinician may be defined based on one or more combinations of: ranges of risk score; risk score value; heart failure with reduced ejection fraction (HfeEF) versus preserved ejection fraction (HFpEF); a patient similarity measure (e.g. cosine similarity, clustering methods, a predefined distance measure, such as Euclidean distance); patient outcomes; and types of treatment received. Again, these noted subgroups are merely illustrative classifications, and are not intended to limit the scope of the present teachings.

At 210, training of a trained feature importance computational model 164 is carried out. As described more fully below, the training of the trained feature importance computational model 164 is based on a pre-defined training data set 180, which includes, for example, the selected subgroup of patients, the input features, the engineered features generated at 204 are provided to the trained feature importance computational model 164 at 206, and the preliminary risk score determined by the trained preliminary risk score computational model 166 at 206. Beneficially, the trained feature importance computational model 164 exhibits an additive property to the risk score generated at 206. Just by way of illustration, the trained feature importance computational model 164 is illustratively the Shapley Value feature importance model, such as described below. In accordance with a representative embodiment, the trained feature importance computational model 164, is illustratively stored in storage 160 as instructions, which when executed by the processor 120, cause the processor to determine the feature importance values using input data comprising the defined patient subgroup, the engineered features and the input features.

At 212, the method continues with the determining of a feature importance value using the trained feature importance computational model 164. As described below in connection with FIG. 3, the feature importance value comprises a contribution of a risk score.

At 214, the method comprises providing decision trajectories for the patient at a particular time (e.g., a critical care point or discharge point), or individualized feature importance values for the patient over time, or both. The decision trajectories are determined using the patient subgroup defined at 208, the engineered features defined at 204 and the preliminary risk score determined at 206.

FIG. 3 flow diagram showing a method 300 of determining decision trajectories and feature contributions, according to a representative embodiment. The method 300 is contemplated to be carried out using patient risk score analysis system 100. As such, and in accordance with various embodiments of the present teachings, the method 300 described herein may be implemented using a hardware computer system such as patient risk score analysis system 100 that executes software programs. Further, in representative, non-limiting embodiments, implementations can include distributed processing, component/object distributed processing, and parallel processing. Virtual computer system processing may implement one or more of the methods or functionalities as described herein, and a processor described herein may be used to support a virtual processing environment. Certain aspects and details of the patient risk score analysis system 100 described in connection with FIG. 1 are common to the method 200 of FIG. 2, and may not be repeated to avoid obscuring the description of the method 200.

The method 300 comprises a first method 302 for providing a preliminary risk score. Many aspects and details of the first method 302 are described in the parent application and may not be repeated herein to avoid obscuring the description of the method 300 of the present teachings. At 304, input features are gathered. As noted above, these input features comprise vital signs such as, for example, blood pressure, temperature, oxygen saturation (SpO2) of the patient.

At 306, the input features are pre-processed and engineered to provide engineered features at 308. The pre-processing and engineering carried out at 306 to provide the engineered features, which provide a clinical composite score for a particular patient, may comprise trends in vital signs over time, a shock ratio, and fluid accumulation, and “unchanged” input data (directly measured data BP, temperature, etc.). Moreover, the engineered features do not include erroneous data points, which are discarded at 306. The engineered features 308 are provided to a trained risk score computational model at 310. This model may be the trained preliminary risk score computational model 166 in storage 160, and includes instructions, which when executed by the processor 120, provide a preliminary risk score at 312.

The method 300 also includes a second method 320 for determining decision trajectories and/or individualized feature contributions across time. Certain aspects and details of the second method 320 are common to the system described in connection with FIG. 1, the method 200 of FIG. 2, and the first method 302. These common aspects and details may not be repeated to avoid obscuring the description of the second method 320.

At 322, patient subgroups are defined. As noted above, there are a variety of subgroups that can be defined to provide better risk assessment in accordance with the present teachings. These can be subgroups that receive different treatment decisions and that the clinical team may want to analyze which treatment decision is best for a given patient by comparing the given patient against patients in the pre-defined subgroups. For example, the subgroups may be established to evaluate a patient against i) low risk patients who are discharged to outpatient settings; and ii) high risk patients who are escalated to more intensive level of care. In accordance with another representative embodiment, subgroups of interest to a clinician may be defined based on one or more combinations of: ranges of risk score; risk score value; heart failure with reduced ejection fraction (HfeEF) versus preserved ejection fraction (HFpEF); a patient similarity measure (e.g. cosine similarity, clustering methods, a predefined distance measure, such as Euclidean distance); patient outcomes; and types of treatment received. Again, these noted subgroups are merely illustrative classifications, and are not intended to limit the scope of the present teachings.

Notably, selecting patient subgroups allows the comparison of a specific patient's decision trajectory against those of the pre-defined subgroups. In this way, it can be determined if a patient's characteristics is similar or different to that of the subgroups' characteristics.

At 324, training of the feature importance computational model is carried out for each individual patient at each point in time over a certain time period. As noted above training data such as training data set 180 are used to train the computational model. As such, training data comprises input features from 304, engineered features from 308 and the preliminary risk score from 310.

Notably, training at 324 of the feature importance computational model is carried out for a pre-defined training set for each patient subgroup. This can be any feature importance metric, and as noted above, is beneficially a method that exhibits an additive property to the risk score (e.g., Shapley Value calculated as described below).

In accordance with a representative embodiment, the training of the feature importance computational model at 324 uses the Shapely Value feature importance method. Specifically, in this representative embodiment, the feature importance is generated per the following:

$${ϕ}_{i} = {Σ}_{S ⊆ F ∖ \{ i \}} \frac{\vert S \vert ! ( \vert F \vert - \vert S \vert - 1 ) !}{\vert F \vert !} [ {f}_{S ⋃ \{ i \}} ( {x}_{S ⋃ \{ i \}} ) - {f}_{S} ( {x}_{S} ) ] \qquad ( Eq . 1 )$$

and the additive property:

g(z′)=ϕ₀+Σᵢ₌₁^{M}ϕᵢz′ᵢ (Eq. 2)

In (Eq. 1) and (Eq. 2), ϕᵢ is the contribution of a given feature (i.e., individualized feature importance); S is the coalition/all subsets of features used for the risk score model; F is the set of all features; f_{S∪{i}} is the model trained with that feature present; f_{S} is the model trained with that feature withheld; x_{S} is the value of the input features in the set D; ϕ₀ is the expected prediction of the model; z′ᵢ∈{0,1}^{M}; and M is the number f of input features. However other methods of generating the personalized feature importance are contemplated.

Beneficially, the additive property of the feature importance values interpretation of the extent the associated feature contributed to the risk score, since the summation of feature importance values across all input features equals the risk score.

It is noted that the trained feature importance computational model 164 described above is merely illustrative. Beneficially, the feature interpretation tools of the present teachings can be applied to a variety of algorithms that receive a set of numerical inputs and compute a numerical output, such as typical machine learning models (e.g. Random Decision Forest, XGoost, etc.), deep learning models (e.g. neural networks, etc.), and non-linear systems. As described above, the selected computational model is illustratively implemented as software that provides AI and applies machine-learning described herein. The AI engine, which provides the computational models described above, may reside in any of various components in addition to or other than storage 160 or memory 130, or both, or in an external server, and/or a cloud, for example.

At 326, using the selected patient subgroup from 322, the engineered features from 308 and the preliminary risk score from 312, the trained feature importance computational model determines decision trajectories at a time of a decision, or individualized feature contributions across time, or both, and provides these as outputs at 328. As described more fully below, the outputs provided at 328 allow clinicians to assess a patient's condition relative to other patients of the subgroup both at a particular time, and over a period of time.

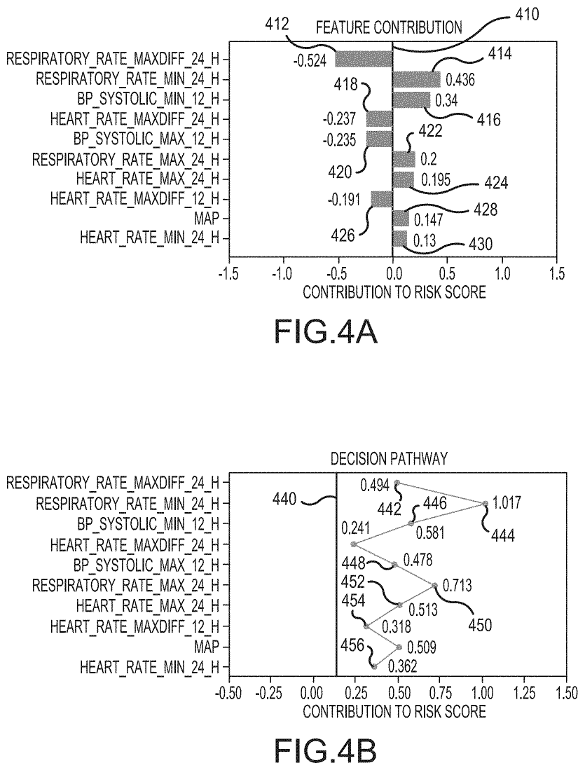

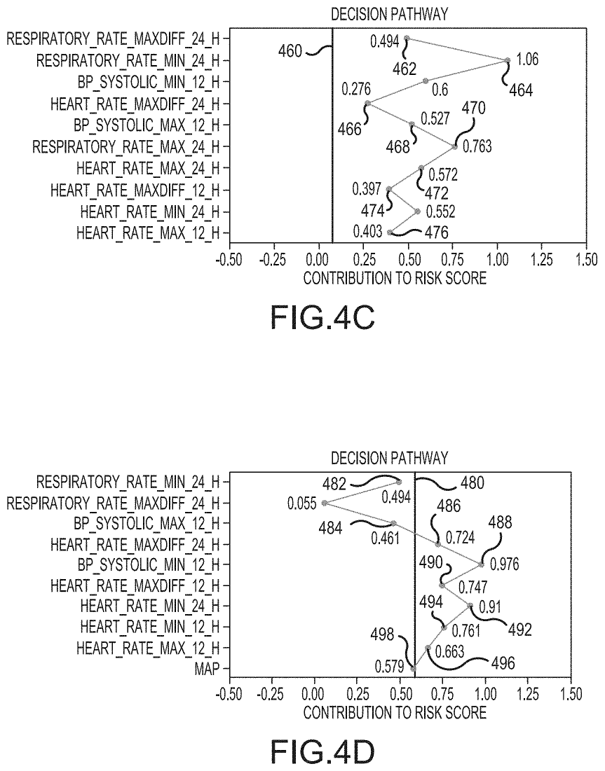

FIGS. 4A-4D are graphs showing feature values and feature contributions at a particular point in time according to a representative embodiment. Certain aspects and details of the graphs depicted in FIGS. 4A-4D are common to the system described in connection with FIG. 1, the method 200 of FIG. 2, and the method 300 of FIG. 3. These common aspects and details may not be repeated to avoid obscuring the description of the graphs of FIGS. 4A-4D.

As described more fully below, the graphs depicted in FIGS. 4A-4D show, for a given point in time, a decision trajectory that shows how input features sum up to the risk score. As alluded to above, features are ranked by their feature importance values for the given patient at the given point in time. Illustratively, the most important feature importance value appears first on top of each graph. In the representative embodiment, the feature importance values for an illustrative top ten contributing features are shown. Moreover, the vertical lines indicate the expected mean risk scores for respective patient subgroups derived from pre-selected training sets.

Turning to FIG. 4A, a graph of risk scores and feature contributions thereto at a particular instant are shown. The data plotted in FIG. 4A are from the output at 328 in FIG. 3.

The vertical line 410 shows the mean risk score of the defined patient group. Bar graphs 412-430 show deviations from the mean feature contribution and risk score for the individual being assessed. For example, at bar graph 412, the patient's maximum difference in respiratory rate over a 24 hour period decreased the risk score by the amount indicated by the length of the bar, the length of the bar represents the contribution away from the expected mean risk score caused by the patient's maximum difference in heart rate over the 24 hour period; and at point 422 the maximum respiratory rate over the 24 hour period is greater than the mean value of this feature contribution, and the length of the bar represents the contribution to the risk score caused by the patient's respiratory rate over the 24 hour period. Notably, the sum of the risk scores of the bar graphs 412-430 provides the risk score for the patient using the trained feature importance computational model described above in connection with FIGS. 1-3. This allows a clinician to assess a patient's risk at a particular instant in time (e.g., at discharge) relative to others in the subgroup.

Turning to FIG. 4B, a graph of risk scores and feature contributions thereto at a particular instant are shown. The data plotted in FIG. 4B are from the output at 328 in FIG. 3.

The vertical line 440 shows the mean risk score of the defined patient group. Data points 442-456 show deviations from the mean feature contribution and risk score for the individual being assessed. For example, at data point 442, the distance on the ordinate represents the contribution to the risk score caused by the patient's maximum difference in respiratory rate over the 24 hour period. Similarly, at point 454 the distance from the vertical line 440 represents the contribution to the risk score caused by the patient's maximum heart rate difference over the 24 hour period over the 24 hour period. Notably, the sum of the risk scores of the data points 4442-456 provides the risk score for the patient using the trained feature importance computational model described above in connection with FIGS. 1-3. This allows a clinician to assess a patient's risk at a particular instant in time (e.g., at discharge) relative to others in the subgroup.

Turning to FIG. 4C, a graph of risk scores and feature contributions thereto at a particular instant is shown. The data plotted in FIG. 4C are from the output at 328 in FIG. 3. Notably, the selected point in time is a decision point on whether a patient should be discharged based on a comparison of other patients in the defined subgroup. In this case, the subgroup of patients are considered to be low risk patients at discharge.

The vertical line 460 shows the mean risk score of the defined patient group. Data points 462-476 show deviations from the mean feature contribution and risk score for the individual being assessed. For example, at data point 462, the length of the bar represents the contribution to the risk score caused by the patient's maximum difference in heart rate over the 24 hour period; and at 466 the maximum respiratory rate over the 24 hour period is greater than the mean value of this feature contribution, and the distance from the vertical line 460 presents the contribution to the risk score caused by the patient's respiratory rate over the over the 24 hour period. Notably, the sum of the risk scores of the data points 462-476 provides the risk score for the patient using the trained feature importance computational model described above in connection with FIGS. 1-3. This allows a clinician to assess a patient's risk at a particular instant in time (e.g., at discharge) relative to others in the subgroup.

Turning to FIG. 4D a graph of risk scores and feature contributions thereto at a particular instant is shown. The data plotted in FIG. 4D are from the output at 328 in FIG. 3. Notably, the selected point in time is a decision point on whether a patient should be discharged based on a comparison of other patients in the defined subgroup. In this case, the subgroup of patients are considered to be high risk patients at discharge.

The vertical line 480 shows the mean risk score of the defined patient group. Data points 482-498 show deviations from the mean feature contribution and risk score for the individual being assessed. For example, at data point 482, the length of the bar represents the contribution to the risk score caused by the patient's minimum difference in respiratory rate over the 24 hour period; and at 488 the maximum heart rate over the 24 hour period is greater than the mean value of this feature contribution, and the distance from the vertical line 460 presents the contribution to the risk score caused by the patient's respiratory rate over the over the 24 hour period. Notably, the sum of the risk scores of the data points 482-496 provides the risk score for the patient using the trained feature importance computational model described above in connection with FIGS. 1-3. This allows a clinician to assess a patient's risk at a particular instant in time (e.g., at discharge) relative to others in the subgroup.

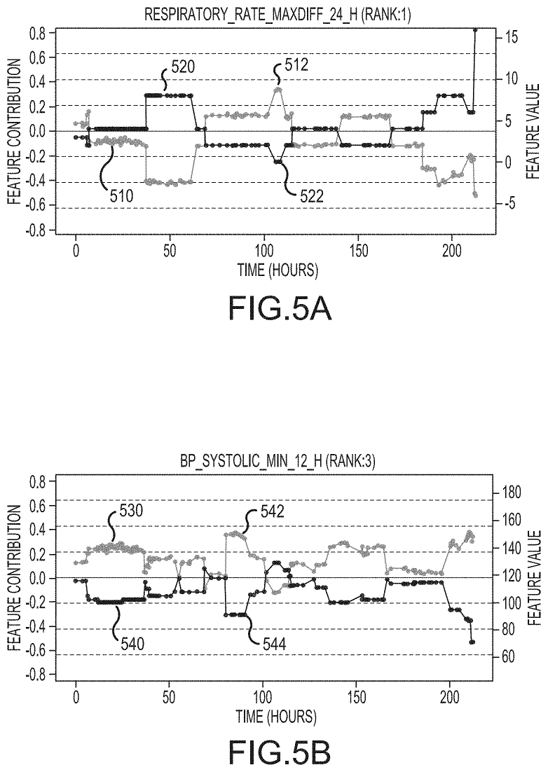

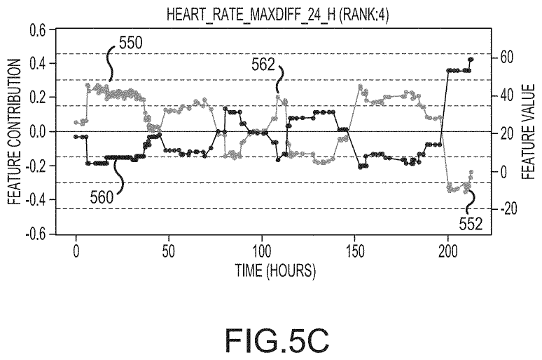

FIGS. 5A-5C are graphs showing feature values and feature contributions at over a particular time period, according to a representative embodiment. To aid interpretation, the different ranks as presented by the dashed horizontal lines indicate a standard deviation of the respective values in the training data set. Certain aspects and details of the graphs depicted in FIGS. 5A-5C are common to the system described in connection with FIG. 1, the method 200 of FIG. 2, and the method 300 of FIG. 3. These common aspects and details may not be repeated to avoid obscuring the description of the graphs of FIGS. 5A-5C.

Turning to FIG. 5A the vertical axis on the right side of the graph depicts the feature value, which is depicted by graph 510. The vertical axis on the left side of the graph depicts the feature contribution, which is depicted by graph 520. The data plotted in graph 510 are from the output at 328 in FIG. 3; and the data plotted in graph 520 are the input features from 304 and the engineered features from 308 in FIG. 3.

As shown, the graph of FIG. 5A is maximum difference in respiratory rate for a particular patient over 200+ hours. Just by way of illustration, it is noted that at point 512, the feature value is approximately 0.35. Notably, the sum of the values on graph 510 equals the risk score. Finally, at point 522, the feature contribution is approximately −0.2.

Turning to FIG. 5B the vertical axis on the right side of the graph depicts the feature value, which is depicted by graph 510. The vertical axis on the left side of the graph depicts the feature contribution, which is depicted by graph 520. The data plotted in graph 510 are from the output at 328 in FIG. 3; and the data plotted in graph 520 are the input features from 304 and the engineered features from 308 in FIG. 3.

As shown, the graph of FIG. 5B is a minimum systolic blood pressure for a particular patient over 200+ hours. Just by way of illustration, it is noted that at point 542, the feature value is approximately 0.35. Notably, the sum of the values on graph 510 equals the risk score. Finally, at point 422, the feature contribution is approximately −0.2.

Turning to FIG. 5C the vertical axis on the right side of the graph depicts the feature value, which is depicted by graph 550. The vertical axis on the left side of the graph depicts the feature contribution, which is depicted by graph 560. The data plotted in graph 550 are from the output at 328 in FIG. 3; and the data plotted in graph 560 are the input features from 304 and the engineered features from 308 in FIG. 3.

As shown, the graph of FIG. 5C is maximum difference in respiratory rate for a particular patient over 200+ hours. Just by way of illustration, it is noted that at point 552, the feature value is approximately −12. Notably, the sum of the values on graph 510 equals the risk score. Finally, at point 522, the feature contribution is approximately −0.2.

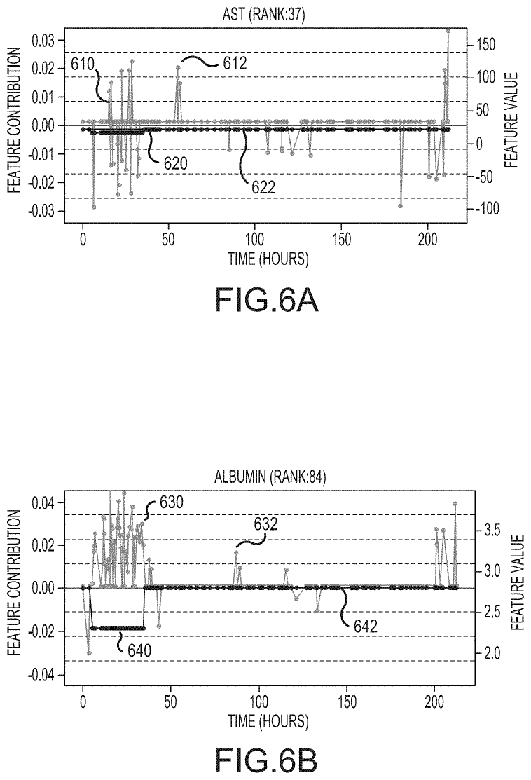

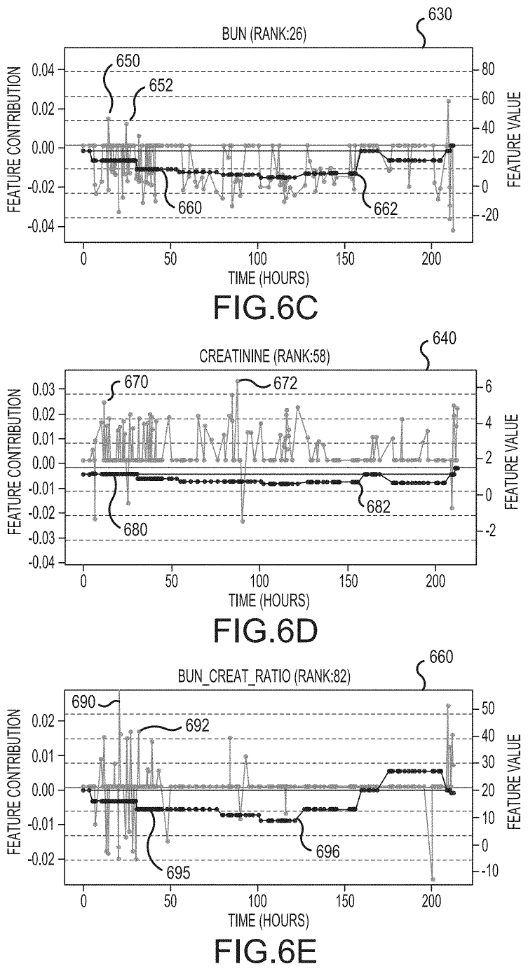

FIGS. 6A-6E are graphs showing feature values and feature contributions at over a particular time period, according to a representative embodiment. Again, to aid interpretation, the different ranks as presented by the dashed horizontal lines indicate a standard deviation of the respective values in the training data set. Certain aspects and details of the graphs depicted in FIGS. 6A-6E are common to the system described in connection with FIG. 1, the method 200 of FIG. 2, the method 300 of FIG. 3, and various aspects and details of FIGS. 4A-4C. These common aspects and details may not be repeated to avoid obscuring the description of the graphs of FIGS. 6A-6C. Notably, the data from graphs of FIGS. 6A-6E are useful in the care of cardiac patients.

Beneficially, the graphs of FIGS. 6A-6E provide temporal information on how a particular patient's clinical measurements change over time and together how these changes proportionally relate to the changes in the risk score. This information provides better support for clinicians in understanding patient's status and response to therapy, and therefore aides in their upcoming clinical decisions.

As shown, the graph of FIG. 6A shows aspartate aminotransferase (AST) for a particular patient over 200+ hours. Just by way of illustration, it is noted that at point 612, the feature value is approximately 0.02. Notably, the sum of the values on graph 610 equals the risk score. Finally, at point 622, the feature contribution is approximately 0.

Turning to FIG. 6B the vertical axis on the right side of the graph depicts the feature value, which is depicted by graph 630. The vertical axis on the left side of the graph depicts the feature contribution, which is depicted by graph 640. The data plotted in graph 630 are from the output at 328 in FIG. 3; and the data plotted in graph 640 are the input features from 304 and the engineered features from 308 in FIG. 3.

As shown, the graph of FIG. 6B depicts albumin levels for a particular patient over 200+ hours. Just by way of illustration, it is noted that at point 632, the feature value is approximately 0.015. Notably, the sum of the values on graph 630 equals the risk score. Finally, at point 642, the feature contribution is approximately 0.

Turning to FIG. 6C the vertical axis on the right side of the graph depicts the feature value, which is depicted by graph 650. The vertical axis on the left side of the graph depicts the feature contribution, which is depicted by graph 660. The data plotted in graph 650 are from the output at 328 in FIG. 3; and the data plotted in graph 660 are the input features from 304 and the engineered features from 308 in FIG. 3.

As shown, the graph of FIG. 6C shows the blood urine nitrogen (BUN) levels for a particular patient over 200+ hours. Just by way of illustration, it is noted that at point 652, the feature value is approximately 0.01. Notably, the sum of the values on graph 650 equals the risk score. Finally, at point 662, the feature contribution is approximately −0.01.

Turning to FIG. 6D the vertical axis on the right side of the graph depicts the feature value, which is depicted by graph 670. The vertical axis on the left side of the graph depicts the feature contribution, which is depicted by graph 680. The data plotted in graph 670 are from the output at 328 in FIG. 3; and the data plotted in graph 680 are the input features from 304 and the engineered features from 308 in FIG. 3.

As shown, the graph of FIG. 6D shows the creatinine levels for a particular patient over 200+ hours. Just by way of illustration, it is noted that at point 672, the feature value is approximately 0.03. Notably, the sum of the values on graph 670 equals the risk score. Finally, at point 682, the feature contribution is approximately −0.005.

Turning to FIG. 6E the vertical axis on the right side of the graph depicts the feature value, which is depicted by graph 690. The vertical axis on the left side of the graph depicts the feature contribution, which is depicted by graph 695. The data plotted in graph 690 are from the output at 328 in FIG. 3; and the data plotted in graph 695 are the input features from 304 and the engineered features from 308 in FIG. 3.

As shown, the graph of FIG. 6E shows the creatinine to BUN ratio for a particular patient over 200+ hours. Just by way of illustration, it is noted that at point 692, the feature value is approximately 0.011. Notably, the sum of the values on graph 690 equals the risk score. Finally, at point 696, the feature contribution is approximately −0.005.

As will be appreciated by one of ordinary skill in the art having the benefit of the present disclosure, the systems and methods of the present teachings provide improvement in the function of an imaging system used to improve monitoring of people in a patient's room or other similar area, and an improvement to the technical field of monitor imaging. For example, compared to known methods and systems, accurate pose estimation and movement of people is realized by the present teachings, at a comparatively high inference speed yet requiring fewer computing resources to known feature extraction methods. Notably, the benefits are illustrative, and other advancements in the field of pose estimation and monitoring of people will become apparent to one of ordinary skill in the art having the benefit of the present disclosure.

Although methods, systems and components for decision making by clinicians are described in connection with various representative embodiments, it is understood that the words that have been used are words of description and illustration, rather than words of limitation. Changes may be made within the purview of the appended claims, as presently stated and as amended, without departing from the scope and spirit of the present teachings.

The illustrations of the representative embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of interpreting a risk score and comparing a decision trajectory, the method comprising:
providing input features comprising clinical measurements of a patient;
generating engineered features from the input features;
providing the engineered features to a trained risk score computational model to determine a preliminary risk score of the patient;
defining a subgroup of patients;
training a feature importance computational model using the subgroup of patients, the engineered features, and the preliminary risk score;
determining a feature importance value using the trained feature importance computational model, the feature importance value comprising a contribution of a risk score; and
providing decision trajectories for the patient, or individualized feature importance values or feature contributions for the patient over time, or both.

2. The method of claim 1, wherein the input features comprise measurements for the patient.

3. The method of claim 2, wherein the measurements comprise one or more vital signs for the patient.

4. The method of claim 1, wherein the providing engineered features comprises deleting input features that outlay other input features.

5. The method of claim 1, wherein the feature importance computational model comprises an additive property of the risk score.

6. The method of claim 5, wherein the feature importance computational model comprises a Shapley Value feature importance computational model.

7. The method of claim 1, wherein the feature importance value is an average marginal contribution of the feature importance value over a plurality of feature subsets.

8. A system for interpreting a risk score and comparing a decision trajectory, the system comprising:
a memory adapted to store: input features comprising clinical measurements of a patient; engineered features; a trained risk score computational model comprising instructions; a defined subgroup of patients; and a processor, wherein the instructions, when executed by the processor causes the processor to:
train a feature importance computational model using the defined subgroup of patients, the engineered features, and the risk score;
determine a feature importance value using the trained feature importance computational model, the feature importance value comprising a contribution of a risk score; and
provide a decision trajectory for the patient, or individualized feature outcomes for the patient over time, or both.

9. The system of claim 8, wherein the input features comprise measurements for the patient.

10. The system of claim 9, wherein the measurements comprise one or more vital signs for the patient.

11. The system of claim 8, wherein the engineered features do not comprise input features that outlay other input features.

12. The system of claim 8, wherein the feature importance computational model comprises an additive property of the risk score.

13. The system of claim 12, wherein the feature importance computational model comprises a Shapley Value feature importance computational model.

14. The system of claim 12, wherein the feature importance value is an average marginal contribution of the feature importance value over a plurality of feature subsets.

15. A tangible, non-transitory computer readable medium that stores input features comprising clinical measurements of a patient; engineered features; a trained risk score computational model comprising instructions; a defined subgroup of patients; and a processor, wherein the instructions, when executed by the processor causes the processor to:
train a feature importance computational model using a subgroup of patients, the engineered features, and a risk score from the trained risk score computational model;
determine a feature importance value using the trained feature importance computational model, the feature importance value comprising a contribution of a risk score; and
provide a decision trajectory for the patient, or individualized feature outcomes for the patient over time, or both.

16. The tangible, non-transitory computer readable medium of claim 15, wherein the input features comprise measurements for the patient.

17. The tangible, non-transitory computer readable medium of claim 16, wherein the measurements comprise one or more vital signs for the patient.

18. The tangible, non-transitory computer readable medium of claim 15, wherein the engineered features do not comprise input features that outlay other input features.

19. The tangible, non-transitory computer readable medium of claim 15, wherein the feature importance computational model comprises an additive property of a risk score from the trained risk score computational model.

20. The tangible, non-transitory computer readable medium of claim 15, wherein the feature importance computational model comprises a Shapley Value feature importance computational model.
